(19) United States
(12) Patent Application Publication
(10) Pub. No.: US 2021/0172459 A1
(43) Pub. Date: Jun. 10, 2021

(54) CENTRIFUGAL PUMP FOR PROCESSING MOLTEN UREA AND RELATIVE PLANT

(71) Applicant: EUROTECNICA MELAMINE AG, Wollerau (CH)

(72) Inventors: Roberto SANTUCCI, Gorla Maggiore (VA) (IT); Giuseppe DI RUOCCO, Lecco (IT); Alberto DE AMICIS, San Donato Milanese (MI) (IT)

(21) Appl. No.: 17/111,837

(22) Filed: Dec. 4, 2020

(30) Foreign Application Priority Data
Dec. 6, 2019 (IT) 102019000023160

(51) Int. Cl. F04D 29/70; F04D 7/06; F04D 29/58; F04D 7/04; F04D 1/06

(52) U.S. Cl. CPC F04D 29/708; F04D 7/06; F04D 1/06; F04D 7/045; F04D 29/586

(57) ABSTRACT

A centrifugal pump (100) for processing molten urea having: an inlet (102) adapted to receive molten urea at a suction pressure; a delivery outlet (104) adapted to allow an output of molten urea at a delivery pressure greater than the suction pressure; and an intermediate outlet (103) adapted to allow an output of molten urea at an intermediate pressure greater than the suction pressure and lower than the delivery pressure.

The present application claims priority to Italian Patent Application No. 102019000023160 filed on Dec. 6, 2019, the entire contents of which is incorporated by reference.

### DESCRIPTION

### Field of the Invention

The present invention relates to the field of industrial processing of urea, particularly for the production of melamine In more detail, the present invention relates to a centrifugal pump for processing molten urea and a relative plant, in particular for producing melamine with a high-pressure process.

### State of the Art

The production of melamine by urea pyrolysis according to the global reaction (1) is known:

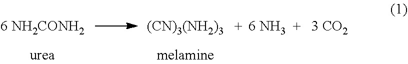

such reaction, as known, being highly endothermic.

The processes for converting urea into melamine are distinguished into two groups: processes performing high-pressure urea pyrolysis and processes performing low-pressure urea pyrolysis.

Both the processes are typically performed in reactors fed with a molten urea stream.

Preferably, the reactor is also fed with an ammonia stream.

In high-pressure processes, the reaction chamber is always held at a pressure greater than 60 bar and it is provided with heating means maintaining the reagent system at a temperature of about 360° C.-450° C.

The present invention relates particularly to processes for the high-pressure preparation of melamine. More specifically, in order to produce melamine, it is necessary to handle and pressurize liquid-state urea with a concentration greater than 99.8%, i.e. molten, taking it either from a vacuum separator, in case the urea is fed to the plant as aqueous solution and needs to be concentrated inside the plant itself, or from a surge drum maintained under a slight pressure, in case the urea is fed to the melamine plant already as concentrated, to a reactor operating at a pressure of about 80 bars.

At present, in order to pressurize the molten urea from the separator to the reactor reciprocating pumps are used. The constant increase of the production capability of plants for producing melamine requested by the market, has determined a proportional increase of urea flow rates to be processed in the plants, resulting in an increase of the volume and number of reciprocating pump heads used, and frequently, in the use of more pumps in parallel, with the result of an increase in the complexity, costs and malfunction risks of the reciprocating pumps currently used with an impact on the operation of the plant itself. The use of reciprocating pumps causes a number of disadvantages.

Reciprocating pumps are by their nature in each head equipped with valves at the inlet (suction) and at the outlet (delivery) that control their operation. The presence of such valves along the pump's suction flow, and in particular at the inlet on each head, is a bottleneck that results in an speed increase and load loss with a subsequent decrease inside the valve itself causing the release of any gases dissolved in the liquid phase with a risk of cavitation phenomena at the pump inlet.

The molten urea is subjected to a continuous, though minimal, decomposition with the formation of ammonia and carbon dioxide, that remain dissolved in the liquid phase under constant pressure conditions, but which are released as a gas phase as the pressure decreases, generating cavitation phenomena that impair the proper operation of the reciprocating pumps reducing their performance and often resulting in mechanical faults. For this reason, reciprocating pumps require the use of an additional pump arranged upstream to cause a pre-compression of the molten urea and preventing cavitation at the reciprocating pump inlet.

Furthermore, the reciprocating pumps have by their nature a pulsating delivery pressure, which requires the use of accumulators downstream the reciprocating pump and other devices capable of damping pulsations and/or operating under pulsating pressures. Moreover, in the production of melamine, this factor affects the reactor subjected to a pulsating feeding which generates the formation of a pulsating reaction gas (see the reaction) with a subsequent internal pulsating pressure and on gases it emits in the pressure control, that also result having a pulsating flow rate, and on the components designated to processing such gases.

Urea has a melting temperature equal to 133° C. In order to avoid urea solidification, the pump needs to be drained and washed after its shutdown. The reciprocating pumps with multiple heads arranged in parallel require that each head to be drained and washed individually, and therefore they require complex draining and washing systems. Such systems are detrimental to the reliability of the pump, increasing the possibility of leaks and clogging, and making these operations particularly time-consuming for the operators who could omit complete execution of the same.

Moreover, the starting of the reciprocating pumps causes a sudden pressure increase in the delivery of the pump itself, which risks to cause malfunctions or damages to the downstream components, particularly to the shut off/check valve, which keeps the reactor under pressure during the urea pre-feeding step wherein the pressure of the reactor is maintained by a constant supply of ammonia. In order to overcome this problem generally the delivery is pre-pressurized in advance, with a consequent increase in the complexity of the plant and of its starting procedures.

In addition, some plants require the presence of spare pumps arranged in parallel to the main pump and kept in stand-by, ready to be started if the running pump will stop. In order to maintain the spare pump in stand-by it is necessary to keep it empty and continuously flushed with steam to ensure that internally, in case of leakage of the shut-off valves, urea cannot accumulate, and with its subsequent degradation resulting in a blockage. As an alternative, it is necessary to pre-fill the stand-by pump with molten urea, guaranteeing a circulation inside it to avoid solidifications. In reciprocating pumps, establishing a recirculation inside the pump is complicated and difficult to obtain in a uniformly distributed manner due to the multiple heads which are not individually flushed, unless a complex circulation system is created for each individual head, thus resulting in increased operational operations and a high number of possible leakage points of urea outwards.

### SUMMARY OF THE INVENTION

A general object of the present invention is to improve the prior art with reference to one or more points of view.

Particularly, an object of the present invention is to overcome the above disadvantages by using a centrifugal pump for processing molten urea and a relative plant, in particular for producing melamine with a high-pressure process.

More in detail, object of the present invention is to provide a centrifugal pump for processing molten urea and a relative plant capable of processing a higher amount of molten urea than the prior art, particularly a centrifugal pump having the prevalence conditions necessary to feed molten urea to the production reactor.

A further object of the present invention is to provide a centrifugal pump for processing molten urea and a relative plant which is less complex than what is currently available.

A further object of the present invention is to provide a centrifugal pump for processing molten urea and a relative plant having less malfunction risks and lower wear of components.

Such objectives are substantially achieved due to what is reported in the attached claims forming integral part of the present description.

According to a first aspect, the present invention relates to a centrifugal pump for processing molten urea having:
an inlet adapted to receive molten urea at a suction pressure;
a delivery outlet adapted to allow an output of molten urea at a delivery pressure greater than the suction pressure;
an intermediate outlet adapted to allow an output of molten urea at an intermediate pressure greater than the suction pressure and lower than the delivery pressure.

According to a second aspect, the present invention relates to a plant for processing molten urea, in particular for producing melamine with a high-pressure process, comprising:
a separator for molten urea having a primary inlet, a recirculation inlet and an outlet for molten urea;
at least one centrifugal pump;

wherein the inlet of the centrifugal pump is fluidly connected to the outlet for molten urea of the separator to receive molten urea at the inlet coming from the separator;

wherein the recirculation inlet of the separator is fluidly connected to the intermediate outlet of the centrifugal pump to receive recirculation molten urea at the inlet coming from the centrifugal pump.

### BRIEF DESCRIPTION OF THE DRAWINGS

The present invention will be clearer from the following detailed description to be considered together with the accompanying drawings wherein:

FIG. 1 shows a schematic view of a plant for processing molten urea according to the present invention;

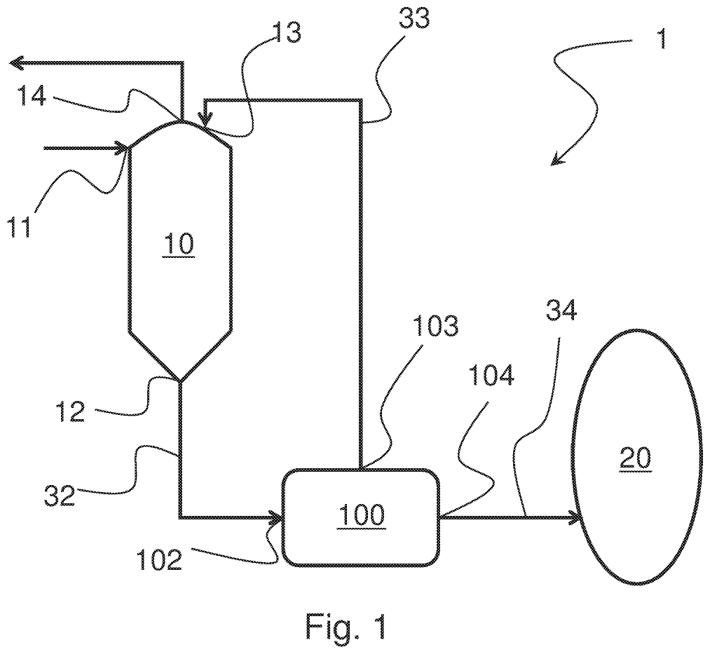

FIG. 2 shows a schematic view of a centrifugal pump for processing molten urea according to the present invention;

FIG. 3 shows a schematic view of a detail of a plant for processing molten urea according to a possible embodiment of the present invention.

FIG. 4 shows a schematic view of a detail of a plant for processing molten urea with the possibility of internal circulation.

FIG. 5 shows a schematic view of a detail of a plant for processing molten urea with flushing possibility.

As easily understandable, there are several modes to implement in practice the present invention which is defined in its main advantageous aspects in the accompanying claims and is not limited either by the following detailed description or by the accompanying drawings.

### DETAILED DESCRIPTION OF THE INVENTION

With reference to the attached figures, a plant for processing molten urea, object of the present invention, is generically indicated with the reference number 1 and hereinafter reference to it will be made with the annotation “plant 1”.

Plant 1 comprises a vacuum separator 10 for molten urea provided with a primary inlet 11, an outlet for molten urea 12, a recirculation inlet 13 and an outlet for gas 14. Preferably, the recirculation inlet 13 is arranged at or in the proximity of an upper end of the separator 10 and the outlet for molten urea 12 is arranged at or in the proximity of a lower end of the separator 10.

The plant 1 also comprises a centrifugal pump for processing molten urea, which forms a further object of the present invention and hereinafter it will be indicated with the reference number 100 and named as “centrifugal pump 100”.

The centrifugal pump 100 has an inlet 102 and a delivery outlet 104. The inlet 102 corresponds to the suction of the centrifugal pump 100 and, when operated, is fluidly connected to the outlet for molten urea 12 of the separator 10 to receive molten urea from it through a suction duct 32. The delivery outlet 104 corresponds to the delivery of the centrifugal pump 100 and, when operated, is fluidly connectable to a reactor 20 for the production of melamine from urea, in order to feed it with a flow rate of molten urea through a delivery duct 34.

The centrifugal pump 100 is configured to receive molten urea at a preselected suction pressure at the inlet 102 and allow an output of molten urea at a delivery pressure at the delivery outlet 104, greater than the suction pressure. Preferably, the suction pressure is comprised between 0.5 bar and 5 bar, even more preferably between 1 bar and 2 bar, particularly of about 1.5 bar. Such suction pressure is determined by the greater height above ground level of the separator 10 (which operates under conditions of absolute vacuum or slight overpressure) with respect to the inlet 102. The difference in height between the separator 10 and the pump inlet 102 must therefore be such as to guarantee a positive pressure in any case.

The delivery pressure is preferably comprised between 60 bar and 100 bar, even more preferably between 70 bar and 90 bar, particularly of about 81 bar. Accordingly, the centrifugal pump 100 has a prevalence preferably comprised between 60 bar and 100 bar, even more preferably between 70 bar and 90 bar, particularly of about 80 bar.

Preferably, the centrifugal pump 100 is configured to process a flow rate of molten urea greater than 15 m³/h, even more preferably greater than 25 m³/h.

Preferably, the centrifugal pump 100 comprises a heating system configured to maintain an internal temperature of the molten urea greater than a preselected value, particularly greater than its melting temperature, equal to about 133° C., in order to avoid solidification inside the centrifugal pump 100 itself.

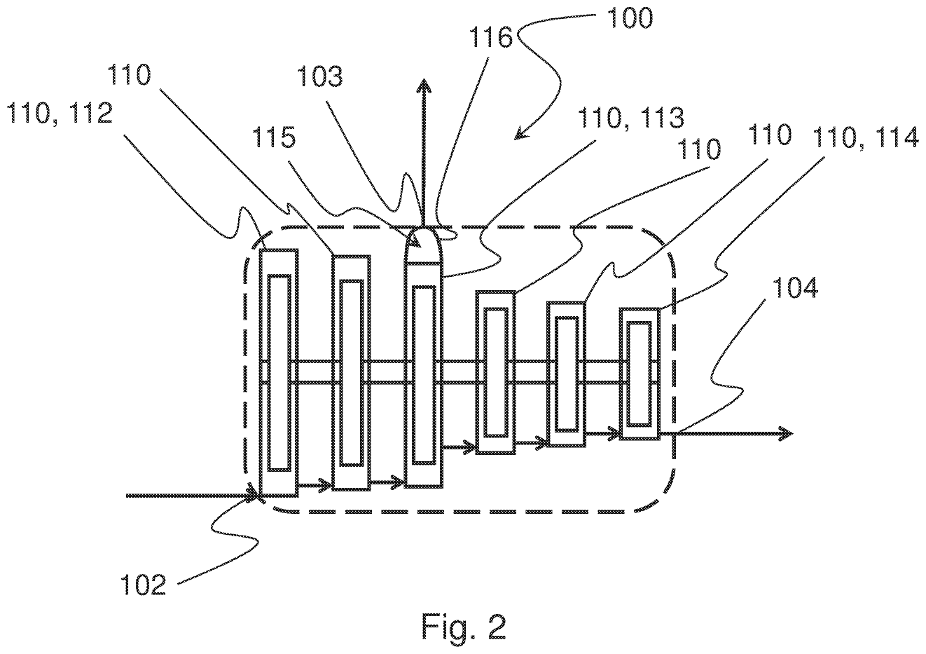

Preferably, the centrifugal pump 100 is multi-stage and comprises a plurality of centrifugal stages 110 serially arranged. Generally, each centrifugal stage 110 has a compression chamber and an impeller arranged inside it, as shown in FIG. 2.

Particularly, the plurality of centrifugal stages 110 includes an initial stage 112 arranged at the inlet 102 to receive molten urea through it and a final stage 114 arranged at the delivery outlet 104 to send molten urea to the reactor 20 through it.

Between the initial stage 112 and the final stage 114 the pressure of the molten urea gradually increases passing through the various centrifugal stages 110.

The plurality of centrifugal stages 110 comprises an intermediate pressure stage 113, operatively serially arranged between the initial stage 112 and the final stage 114.

Particularly, the intermediate-pressure stage 113 has an intermediate outlet 103 configured and arranged to allow the output of molten urea from such stage at an intermediate pressure greater than the suction pressure and lower than the delivery pressure. Preferably, the intermediate pressure is comprised between 3 bar and 15 bar, even more preferably between 5 bar and 10 bar. In other words, the intermediate pressure is greater than the suction pressure by a value comprised between 2 bar and 14 bar, more preferably between 4 bar and 9 bar.

The value of said intermediate pressure depends on the serially arrangement of the intermediate-pressure stage 113 in the plurality of centrifugal stages 110. Preferably, the intermediate-pressure stage 113 is a centrifugal stage interposed between the initial stage 112 and the final stage 114. In some embodiments, the stage at intermediate pressure stage 113 can coincide with the initial stage 112.

Preferably, the intermediate outlet 103 is sized and configured to output a flow rate of molten urea comprised between 50% and 75% of the molten urea at the inlet, even more preferably between 60% and 70%, particularly about 67%.

Moreover, the intermediate outlet 103 when operated, is preferably located at an upper end of the intermediate-pressure stage 113 to allow a gas extraction or venting. The molten urea continuously generates reaction gases, particularly ammonia and carbon dioxide, and the intermediate-pressure stage 113 thus configured allows to convey the gases generated from urea during the movement through the intermediate outlet 103 with operating advantage of the subsequent compression stages.

Preferably, the intermediate-pressure stage 113 has a storage volume 115 arranged, an operative configuration, above the impeller, in fluid communication with the compression chamber. Particularly, the centrifugal pump 100 has a casing provided with a dome-shaped portion 116 vertically above the impeller of the intermediate-pressure stage 113 to define the above-mentioned storage volume 115. The intermediate outlet 103 is arranged at an upper end of the dome-shaped portion 116.

The storage volume 115 is configured to receive molten urea and gas from the compression chamber of the intermediate-pressure stage 113 during operation of the centrifugal pump 100 to allow its input through the intermediate outlet 103.

The intermediate outlet 103 is, when operated, fluidly connected to the separator 10 through a recirculation duct 33, such that to cause a recirculation stream of molten urea from the centrifugal pump 100 to the separator 10 such as to prevent accumulation of solid residues inside it. Particularly, the recirculation duct 33 is fluidly connected to the recirculation inlet 13 of the separator 10. The feed duct 32 is connected to the outlet for molten urea 12. This configuration allows to determine a continuous flow of molten urea from the recirculation inlet 13 to the outlet for molten urea 12 through the entire extension of the separator 10.

The plant 1 further comprises the reactor 20 for producing melamine fluidly connected to the delivery outlet 104 of the centrifugal pump 100 to receive from it pressurized molten urea through the delivery duct 34.

According to a possible embodiment, the centrifugal pump 100 has a variable speed to regulate the flow rate of the molten urea directed to the reactor 20.

According to a possible alternative embodiment, the centrifugal pump 100 has a fixed rotation speed and the plant 1 comprises a regulation valve arranged along the delivery duct 34, between the delivery outlet 104 of the centrifugal pump 100 and the reactor 20, to regulate the flow rate of the molten urea directed to the reactor 20.

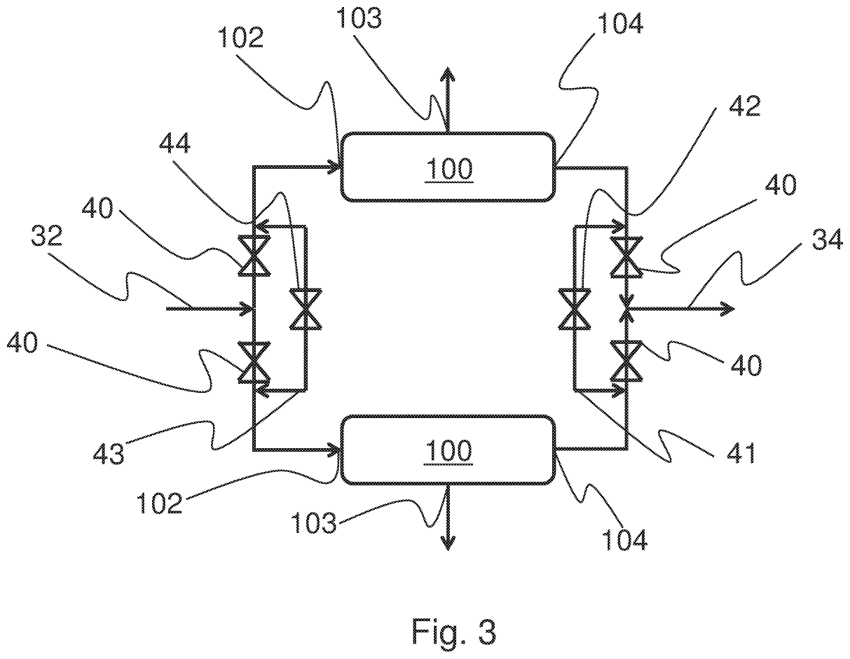

Preferably, plant 1 comprises two of the above-described centrifugal pumps 100, arranged in parallel for redundancy, as illustrated in FIG. 3, to compress the molten urea directed from the separator 10 to the reactor 20. One of the two centrifugal pumps 100 is configurable in stand-by while the other is running

A plurality of valves 40 is arranged along the suction and delivery ducts of the two valves to allow the centrifugal pump 100, when operated, to be connected in line between the separator 10 and the reactor 20 and to isolate the other. Preferably, plant 1 further comprises a first flushing line 41 controlled by a related valve 42 and arranged to cause a fluid coupling between the delivery outlets 104 of the two centrifugal pumps 100, bypassing the valves 40. Moreover, plant 1 can comprise a second flushing line 43 controlled by a related valve 44 and arranged to cause a fluid coupling between the inlets 102 of the two centrifugal pumps 100, bypassing the valves 40.

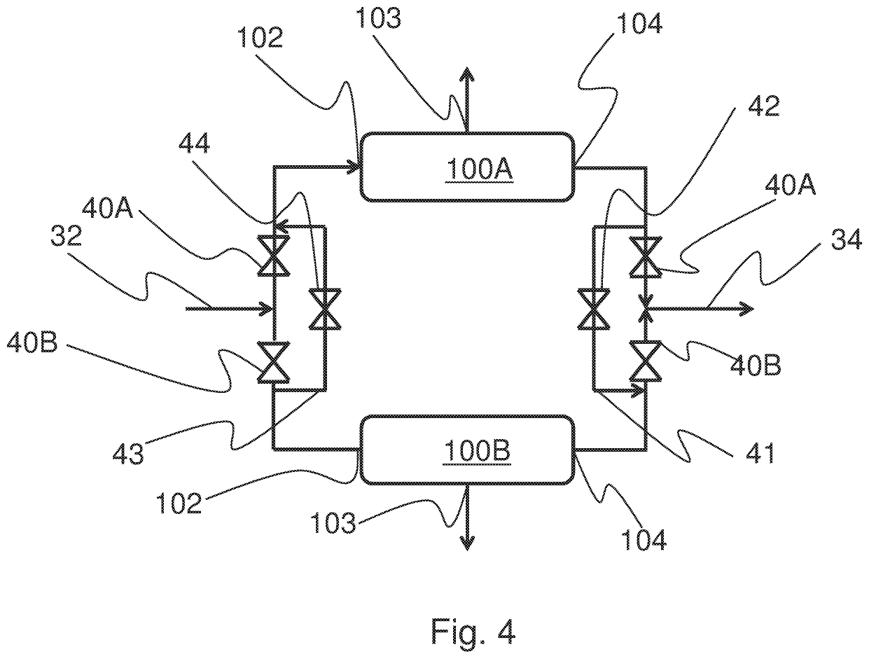

FIG. 4 shows an example of an operational configuration of the plant 1 wherein the centrifugal pump 100A is running, the centrifugal pump 100B is in stand-by, the valves 40A, 42 and 44 are open and the valves 40B are closed.

In the absence of the second flushing line 43, the re-circulation of the fluid in the centrifugal pump 100B in stand-by can be obtained by leaving open or partially open the valve 40B upstream (or in suction) of the centrifugal pump 100B in stand-by.

Advantageously, the first and the second flushing line 41 and 43 allows to establish, during the operation of one of the two centrifugal pumps 100, a flow of molten urea from the delivery outlet 104 of the running centrifugal pump 100 to the inlet 102 of the other centrifugal pump 100 in stand-by, circulating inside the pump in stand-by, and reaching the suction line 102 of the running pump.

By means of this internal circulation in the stand-by pump it is avoided that molten urea can stay inside it with the possibility of decomposition and solidification.

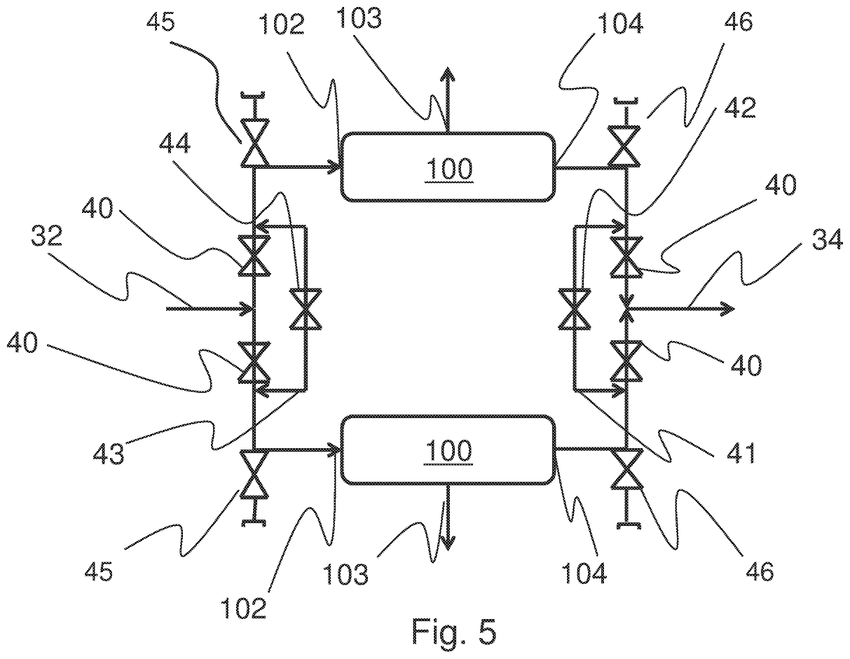

Preferably, plant 1 further comprises a system for washing the one or more centrifugal pumps 100 after their shutdown, an example of which is given in FIG. 5.

The washing of the centrifugal pumps 100 consists in emptying them from the molten urea by draining them into a recovery manifold, followed by flushing with steam and subsequently with water which are in turn recovered to avoid environmental pollution. For this purpose, plant 1 comprises a plurality of access portions 45 and 46, fluidly coupled to the centrifugal pumps 100 to allow their emptying and flushing. Particularly plant 1 comprises a prima access portion 45 upstream of the centrifugal pump 100 and a second access portion 46 downstream of each centrifugal pump 100. Preferably, the first and second access portions 45 e 46 consist of rapid connections equipped with valves, which allow the connection of drainage and/or flushing ducts to perform these operations.

These first and second access portions 45 and 46 allow access to any areas where the molten urea may have solidified, which tends to solidify in the dead points, i.e. without circulation and renewal of the product, for example the areas upstream of the rapid connection valves.

The described embodiments overcome the limitations of the prior art.

The use of the centrifugal pump with these characteristics in place of the traditional reciprocating pumps allows to process a greater amount of molten urea without having to adopt a large number of reciprocating heads or pumps in parallel.

The use of the centrifugal pump with these characteristics prevents the phenomena of cavitation in the suction and allows not to use additional pumps to pre-compress the molten urea.

The described centrifugal pump allows to send a recirculation flow rate at right pressure to the vacuum separator, if present, in order to prevent the accumulation of solid residues without requiring a dedicated pump.

The described centrifugal pump also allows to avoid problems caused to the plant by the delivery pulsating pressure of the reciprocating pumps.

The use of the described solutions makes it possible to create simpler plants having less malfunction risks and lower wear of components.

Comparing the construction and operating peculiarities of centrifugal pumps with respect to reciprocating pumps, the greater simplicity of washing the former compared to the latter is clear, considering that:
there is only one pumping body and not a plurality of in parallel bodies with the possibility of by-passing by the washing fluid or urea retention during the drainage phase, which make the operation unsafe;
they can be flushed in both directions, lacking in the internal check valves present in the reciprocating pumps.

1. A centrifugal pump for processing molten urea having:
an inlet adapted to receive molten urea at a suction pressure;
a delivery outlet adapted to allow an output of molten urea at a delivery pressure greater than said suction pressure;
an intermediate outlet adapted to allow an output of molten urea at an intermediate pressure greater than said suction pressure and lower than said delivery pressure.

2. The centrifugal pump according to claim 1, comprising a plurality of centrifugal stages arranged in series;
said plurality of centrifugal stages comprising:
an intermediate pressure stage arranged and/or configured to cause a molten urea pressure equal to said intermediate pressure;
said intermediate outlet being arranged at said intermediate pressure stage to receive molten urea from said intermediate pressure stage.

3. The centrifugal pump according to claim 2, wherein said plurality of centrifugal stages comprises:
an initial stage arranged at said inlet to receive molten urea from said inlet;
a final stage arranged at said delivery outlet to send molten urea to said delivery outlet;
said intermediate pressure stage being operatively arranged between said initial stage and said final stage.

4. The centrifugal pump according to claim 2, wherein said intermediate outlet is arranged at an upper end of said intermediate pressure stage to allow a gas extraction from said intermediate pressure stage through said intermediate outlet.

5. The centrifugal pump according to claim 4, wherein said intermediate pressure stage has a compression chamber, an impeller arranged in said compression chamber and a storage volume arranged above said impeller, in fluid communication with said compression chamber;
said storage volume being configured to receive molten urea and gas from said compression chamber during an operation of the centrifugal pump and said intermediate outlet being arranged at an upper end of said storage volume.

6. The centrifugal pump according to claim 5, comprising a casing provided with a dome-shaped portion, said dome-shaped portion defining superiorly said storage volume.

7. The centrifugal pump according to claim 1, comprising a heating system adapted to maintain a temperature of the molten urea inside the centrifugal pump greater than a predetermined value.

8. The centrifugal pump according to claim 1, adapted to process a flow rate of molten urea greater than 15 m̂3/h, wherein said delivery pressure is greater than said suction pressure by a value comprised between 50 bar and 100 bar.

9. The centrifugal pump according to claim 1, wherein said intermediate pressure is greater than said suction pressure by a value comprised between 2 bar and 14 bar, and wherein said centrifugal pump is configured to direct a flow rate of molten urea comprised between 50% and 75% of the molten urea at the inlet through said intermediate outlet.

10. A plant for processing molten urea, in particular for producing melamine with a high-pressure process, comprising:
a separator for molten urea having a primary inlet, a recirculation inlet and an outlet for molten urea;
at least one centrifugal pump according to claim 1;
wherein the inlet of the centrifugal pump is fluidly connected to the outlet for molten urea of the separator to receive molten urea at the inlet coming from said separator;
wherein the recirculation inlet of the separator is fluidly connected to the intermediate outlet of the centrifugal pump to receive recirculation molten urea at the inlet coming from said centrifugal pump.

11. The plant according to claim 10, comprising a reactor for producing melamine in a high-pressure process, fluidly connected to the delivery outlet of said centrifugal pump to receive pressurized molten urea from said centrifugal pump.

12. The plant according to claim 11, wherein
said centrifugal pump has a variable speed to regulate a flow rate and/or a pressure of the molten urea directed to said reactor; or
said centrifugal pump has a fixed rotation speed and said plant comprises a regulation valve operatively arranged between the delivery outlet of said centrifugal pump and said reactor to regulate a flow rate and/or a pressure of the molten urea directed to said reactor.

13. The plant according to claim 10, wherein said at least one centrifugal pump comprises two centrifugal pumps,
said two centrifugal pumps being arranged in parallel;
one of said two centrifugal pumps being configurable in stand-by while the other is running.

14. The plant according to claim 13, comprising a first flushing line adapted to cause a fluid coupling between the delivery outlets of said two centrifugal pumps and a second flushing line adapted to cause a fluid coupling between the inlets of said two centrifugal pumps;
the running centrifugal pump being adapted to cause a flow of molten urea from the delivery outlet to the inlet of said centrifugal pump in stand-by through said first and second flushing line in an operating condition of said plant.

15. The plant according to claim 10, comprising at least one first access portion fluidly coupled to said at least one centrifugal pump upstream of said at least one centrifugal pump and at least one second access portion fluidly coupled to said at least one centrifugal pump downstream of said at least one centrifugal pump;
said first and second access portions being adapted to drain and/or flush said at least one centrifugal pump.

16. The centrifugal pump according to claim 8, adapted to process a flow rate of molten urea greater than 25 m̂3/h.

17. The centrifugal pump according to claim 8, wherein said delivery pressure is greater than said suction pressure by a value comprised between 70 bar and 90 bar.

18. The centrifugal pump according to claim 16, wherein said delivery pressure is greater than said suction pressure by a value comprised between 70 bar and 90 bar.

19. The centrifugal pump according to claim 9, wherein said intermediate pressure is greater than said suction pressure by a value comprised between 4 bar and 9 bar.

20. The centrifugal pump according to claim 9, wherein said centrifugal pump is configured to direct a flow rate of molten urea comprised between 60% and 70%, through said intermediate outlet.
